(19) United States
(12) Patent Application Publication
(10) Pub. No.: US 2017/0340513 A1
(43) Pub. Date: Nov. 30, 2017

(54) MASSAGE DEVICE

(71) Applicant: WELLMIKE TECHNOLOGY CORPORATION, New Taipei City (TW)

(72) Inventor: Chung-Yang CHEN, New Taipei City (TW)

(21) Appl. No.: 15/207,748

(22) Filed: Jul. 12, 2016

(30) Foreign Application Priority Data
May 26, 2016 (TW) 105116435

(51) Int. Cl. A61H 39/04; A61N 5/06; A61H 1/00

(52) U.S. Cl. CPC A61H 39/04; A61H 1/008; A61N 5/0619; A61H 2201/10; A61H 2201/1207; A61N 2005/0651; A61H 2201/50

(57) ABSTRACT

A massage device includes a device body for sleeving a finger or a toe, and a massage assembly located at the device body and further including a power element, a plurality of light-emitting elements and a plurality of transparent protrusive massage bodies. The power element located inside the device body is to provide a power for moving the device body. Each of the light-emitting elements is located on a corresponding light path of a light beam emitted by the corresponding light-emitting element. When the device body sleeves the finger or the toe, the transparent protrusive massage bodies contact the finger or the toe at one acupuncture point at least. When the device body is moved, the transparent protrusive massage bodies massage mechanically the at least one acupuncture point, and the light beam penetrating the corresponding transparent protrusive massage body is to massage optically the at least one acupuncture point.

This application claims the benefit of Taiwan Patent Application Serial No. 105116435, filed May 26, 2016, the subject matter of which is incorporated herein by reference.

### BACKGROUND OF INVENTION

### 1. Field of the Invention

The invention relates to a massage device, and more particularly to the massage device that can apply both a mechanical massage and an optical massage upon acupuncture points simultaneously; in which the mechanical massage is performed through a transparent protrusive massage body, and the optical massage is performed through light beams penetrating the transparent protrusive massage body.

### 2. Description of the Prior Art

With the technology progresses rapidly day by day, modern 3C products such as the smart phones and the tablet computers have become one of the indispensable tools in ordinary life. However, as the daily usage time of the aforesaid electronic products extends, a problem of eye fatigue is raised as people stay a long time on the screen or display of the electronic products.

Thus, to ease the critical problem of eye fatigue, the eye care becomes one of important items to be carried out every day. Recently, as the acupressure (i.e. massage at acupuncture points) prevails, an effective treatment around the eyes by the acupressure is proposed to alleviate a little bit the eye fatigue. In the art, the acupressure is performed at specific acupuncture points by a massage master, the effect and depth of the acupressure at individual acupuncture point are highly related to the massage master himself/herself. Further, since the conventional acupressure is performed between skins, thus a long-term application of the acupressure would definitely damage finger knuckles of the massage master.

Hence, related manufacturers propose electric massage devices to perform the acupressure. However, though the maturity at the technology of optical massage is already there, yet current massage devices can implement massage blocks only to process the mechanical massage, but can't extend the performance by including the optical massage at the same time. Thus, it is definitely welcome to the art that an acupressure apparatus can perform the mechanical massage and the optical massage simultaneously.

### SUMMARY OF THE INVENTION

In viewing that the conventional massage device can provide the mechanical massage only and can't provide the optical massage at the same time, thus the massage performance can't be further elevated to an advance stage. Accordingly, it is the primary object of the present invention to provide a massage device that includes a plurality of transparent protrusive massage bodies for performing the mechanical massage to be located on the light path of the light beams from light-emitting elements, such that the optical massage through the transparent protrusive massage bodies can be performed as well.

In the present invention, the massage device includes a device body and a massage assembly. The device body is to sleeve a finger or a toe. The massage assembly located at the device body further includes a power element, a plurality of light-emitting elements and a plurality of transparent protrusive massage bodies. The power element located inside the device body is to provide a power for moving the device body. Each of the light-emitting elements is located on the corresponding light path of the light beam emitted by the corresponding light-emitting element. When the device body sleeves one of the finger and the toe, the plurality of transparent protrusive massage bodies contact the one of the finger and the toe at one acupuncture point at least. When the device body is moved, the plurality of transparent protrusive massage bodies massage mechanically the at least one acupuncture point, and the light beam penetrates the corresponding transparent protrusive massage body to massage optically the at least one acupuncture point.

In one embodiment of the present invention, the transparent protrusive massage body includes a lens structure, and the light-emitting element is an LED (light-emitting diode). In addition, the massage assembly further includes a light-emitting element drive circuit electrically coupled with the light-emitting elements to have the light-emitting elements to emit the corresponding light beams. In addition, the massage device further includes a processing unit located inside the device body and electrically coupled with the power element and the light-emitting element drive circuit. The processing unit further includes a massage program, and the processing unit bases on the massage program to trigger the light-emitting element drive circuit to have the light-emitting elements to emit the light beams and to have the power element to provide a power for moving the device body. The processing unit is a micro controller unit (MCU).

In one embodiment of the present invention, the massage device further includes a USB (Universal serial bus) port, a battery and a power-managing module. The USB port located at the device body is electrically coupled with an external power source for providing a first power source. The battery located inside the device body is to store a secondary power source. The power-managing module located inside the device body is electrically coupled with the USB port. The battery and the processing unit transmit at least one of the first power source and the secondary power source to the processing unit, and further transmit the first power source to the battery to be stored as the secondary power source. In addition, the massage device further includes a trigger element located at the device body and electrically coupled with the power-managing module. When the trigger element is triggered, the power-managing module transmits at least one of the first power source and the secondary power source to the processing unit, so that the processing unit triggers the light-emitting element drive circuit to have the light-emitting elements to emit the light beams and to have the power element to move the device body. The trigger element is a button.

In one embodiment of the present invention, the device body includes a plurality of indicators electrically coupled with the processing unit, and the processing unit bases on the massage program to control the indicators. In addition, the device body has two through holes located individually to two opposing sides of the device body. The through hole allows a fastening belt to pass therethrough so as thereby to fix the device body onto one of the finger and the toe, such that the transparent protrusive massage bodies can contact the at least one acupuncture point of the finger or the toe. In addition, the massage assembly further includes a linkage mechanism connecting the power element and the transparent protrusive massage bodies. When the power element provides the power, the linkage mechanism moves the transparent protrusive massage bodies synchronously.

In one embodiment of the present invention, the power element is a motor, and the massage device further includes a USB (Universal serial bus) port, a battery and a power-managing module. The USB port located at the device body is electrically coupled with an external power source for providing a first power source. The battery located inside the device body is to store a secondary power source. The power-managing module located inside the device body is electrically coupled with the USB port, the battery, the power element and the light-emitting elements, so as to transmit at least one of the first power source and the secondary power source to the power element and the light-emitting elements and to transmit the first power source to the battery to be stored as the secondary power source.

In one embodiment of the present invention, the massage device further includes a trigger element located at the device body and electrically coupled with the power-managing module. When the trigger element is triggered, the power-managing module transmits at least one of the first power source and the secondary power source to the power element and the light-emitting elements. The trigger element is a button. In addition, the device body further includes a plurality of indicators electrically coupled with the power-managing module. When the trigger element is triggered, the power-managing module further transmits at least one of the first power source and the secondary power source to the indicators so as to light up the indicators.

By providing the massage device in accordance with the present invention, since the conventional massage blocks have been replaced by the transparent protrusive massage bodies and the light-emitting elements are included to emit individual light beams to travel along the corresponding light paths, so a user of the massage device can apply the transparent protrusive massage bodies to mechanically massage the acupuncture points, and also can optically massage these same acupuncture points via the transparent protrusive massage bodies and the corresponding light-emitting elements. Thereupon, the overall massage effort can be significantly increased.

All these objects are achieved by the massage device described below.

### BRIEF DESCRIPTION OF THE DRAWINGS

The present invention will now be specified with reference to its preferred embodiment illustrated in the drawings, in which:

FIG. 1 is a schematic perspective view of a first embodiment of the massage device in accordance with the present invention;

FIG. 2 is another view of FIG. 1;

FIG. 3 is a schematic side view of the transparent protrusive massage bodies and the light-emitting elements for the first embodiment of FIG. 1;

FIG. 4 is a schematic block view of the FIG. 1;

FIG. 5 demonstrates a finger to be cooperated with the massage device of FIG. 1;

FIG. 6 is a schematic side view of the transparent protrusive massage bodies and the light-emitting elements for a second embodiment of the massage device in accordance with the present invention; and

FIG. 7 is a schematic block view of the FIG. 6.

### DESCRIPTION OF THE PREFERRED EMBODIMENT

The invention disclosed herein is directed to a massage device. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

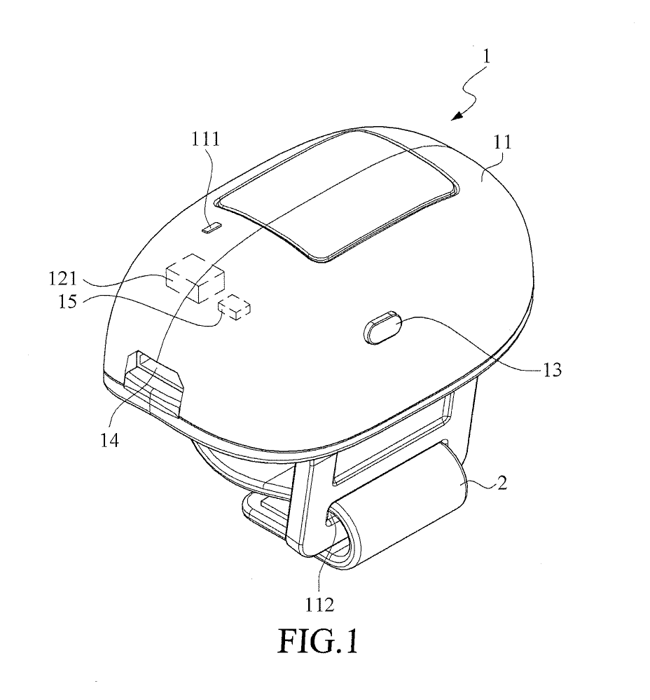

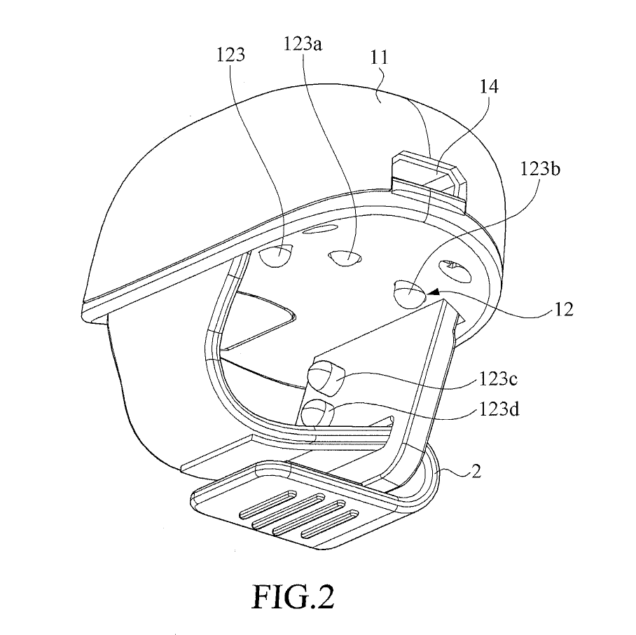

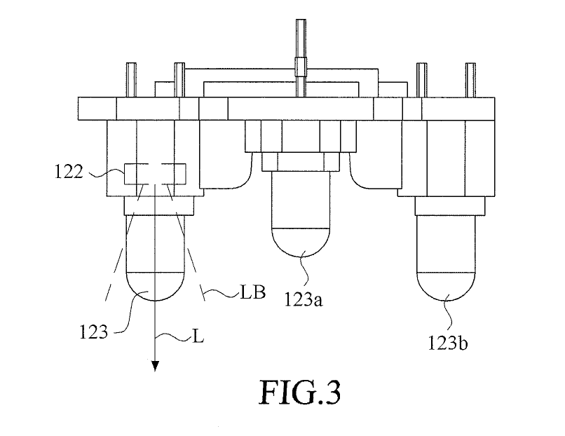

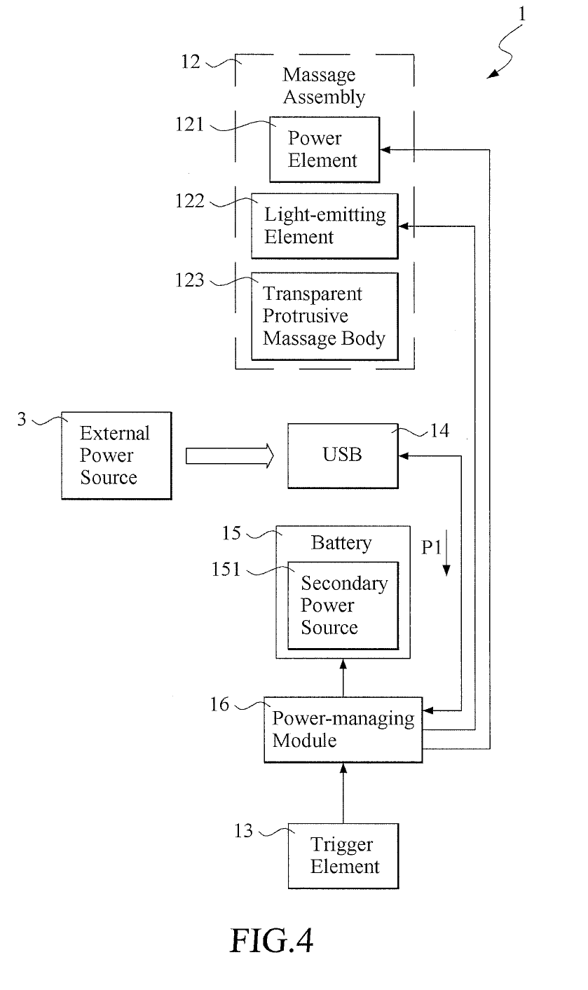

Refer now to FIG. 1 through FIG. 4; where FIG. 1 is a schematic perspective view of a first embodiment of the massage device in accordance with the present invention, FIG. 2 is another view of FIG. 1, FIG. 3 is a schematic side view of the transparent protrusive massage bodies and the light-emitting elements for the first embodiment of FIG. 1, and FIG. 4 is a schematic block view of the FIG. 1.

As shown, in the first embodiment of the present invention, the massage device 1 includes a device body 11, a massage assembly 12, a trigger element 13, a USB (Universal serial bus) port 14, a battery 15 and a power-managing module 16.

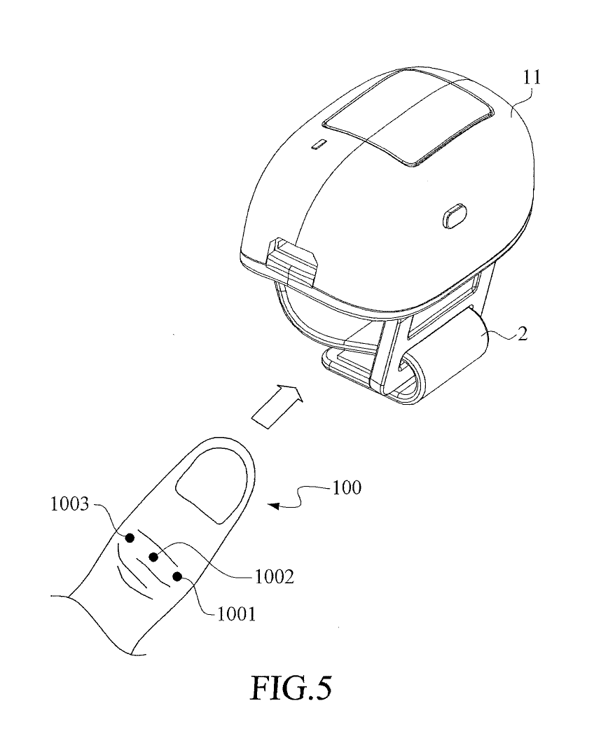

The device body 11 is to sleeve a finger 100 (as a typical example herein) or a toe. In particular, the finger 100 includes at least one acupuncture point (three 1001, 1002 and 1003 shown in FIG. 5). As shown in FIG. 5, the acupuncture point 1001 is corresponding to the eichhornia hole (or called as the second bean hole), the acupuncture point 1002 is corresponding to the big hollow-bone hole (or called as the big bone-cavity hole), and the acupuncture point 1003 is corresponding to the bright-eye hole (or called as the first bean hole). However, it shall be understood the aforesaid acupuncture points 1001, 102 and 103 are only raised for typical examples to elucidate the first embodiment of the massage device in accordance with the present invention, not for limiting the application of the present invention. In some other embodiments of the present invention (not shown herein), other acupuncture points can be still applicable.

The device body 11 has a plurality of indicators 111 (one shown in the figure) and two through holes 112 located individually to two opposing sides of the device body 11. The indicator 111 can be an LED (Light-emitting diode), and the through hole 112 is to allow a fastening belt 2 to pass therethrough so as thereby to fix the device body 11 onto the finger 100 or the toe.

The massage assembly 12 located at the device body 11 includes a power element 121, a plurality of light-emitting elements 122 (seven in the first embodiment, but only three shown in FIG. 3 with only one labeled) and a plurality of transparent protrusive massage bodies 123, 123a, 123b, 123c and 123d (seven in the first embodiment, but only five labeled).

The power element 121 located inside the device body 11 is to provide a power to move the device body 11. In the present invention, the power element 121 can be, but not limited to, a motor.

The light-emitting elements 122 are located individually inside the device body 11. In practice, the device body 11 has apertures (as shown in FIG. 2) to allow the corresponding light-emitting elements 122 to expose partly so as to emit corresponding light beams LB to travel along corresponding light paths L. The light-emitting element 122 can be, but not limited to, an LED or an incandescent bulb. In addition, it shall be noted that, though the light path L shown in FIG. 3 is denoted by a straight line, the light path L can be a radiation-type light path.

The transparent protrusive massage bodies 123, 123a, 123b, 123c and 123d are individually mounted to the corresponding light paths L (in a one-to-one manner) of the light beams LB emitted by the corresponding light-emitting elements 122. Practically, in this first embodiment, the transparent protrusive massage body 123 is located on the light path L of the light beam LB emitted by the light-emitting element 122, while another two transparent protrusive massage bodies 123a and 123b are also mounted in correspondence with the other two light-emitting elements 122, respectively. While the device body 11 sleeves the target finger 100 or toe, the transparent protrusive massage bodies 123, 123a and 123b are to contact the acupuncture points 1001, 1002 and 1003, respectively; and the other two transparent protrusive massage bodies 123c and 123d and the other two non-labeled transparent protrusive massage bodies are to contact other acupuncture points (not shown in the figure).

In addition, it shall be noted that, in this first embodiment, the other two non-labeled transparent protrusive massage bodies are located closer to the fastening belt 2 and symmetrically to the transparent protrusive massage bodies 123c and 123d. Practically, as shown in FIG. 2, the device body 11 can have two separate circuit board structures or the like plate bodies extending downward from a bottom (locating the transparent protrusive massage bodies 123, 123a and 123b) thereof, and the transparent protrusive massage bodies 123c and 123d and the associated light-emitting elements 122 are located to one of the circuit boards at an inward side thereof while another two non-labeled transparent protrusive massage bodies and the associated light-emitting elements are located to another circuit board also at an inward side thereof.

In other embodiments, the device body 11 can waive the two circuit-board extensions, but mount the transparent protrusive massage bodies 123c and 123d and the two non-labeled transparent protrusive massage bodies, as well as the associated light-emitting elements 122, directly to the fastening belt 2.

In the aforesaid embodiment of the present invention, the transparent protrusive massage body 123 is to contact the acupuncture point 1001 of the finger 100, the transparent protrusive massage body 123a is to contact the acupuncture point 1002 of the finger 100, and the transparent protrusive massage body 123b is to contact the acupuncture point 1003 of the finger 100. In other embodiments not shown herein, two or more transparent protrusive massage bodies can be applied to contact the same acupuncture point, or a transparent protrusive massage body can be used to contact three acupuncture points at the same time. Varieties of the like applications depend on the practical needs. In addition, the transparent protrusive massage bodies 123c and 123d and the other two non-labeled transparent protrusive massage bodies can be used to contact the other four acupuncture points, and, however, details thereabout would be omitted herein.

The trigger element 13 mounted at the device body 11 can be a button. In other embodiments, the trigger element 13 can be a knob or any activation element. Also, the trigger element 13 can be integrated with a drive circuit or other functional circuits.

The USB port 14 mounted at the device body 11 is to electrically couple an external power source 3 for providing a first power source P1 to energize the massage device 1. Practically, the external power source 3 can be the municipal electricity system.

The battery 15 mounted inside the device body 11 is to store a secondary power source 151. The battery 15 can be a chargeable battery already in the market place, and thus details thereabout would be omitted herein.

The power-managing module 16 located inside the device body 11 is electrically coupled with the indicator 111, the trigger element 13, the USB port 14, the battery 15, the power element 121 and the plurality of light-emitting elements 122. The power-managing module 16 can be consisted of at least one processor with power-managing ability already in the market place. The power-managing module 16 is to transmit at least one of the first power source P1 and the secondary power source 151 to the indicator 111, the power element 121 and the plurality of light-emitting elements 122, and to also transmit the first power source P1 to the battery 15 as the secondary power source 151.

Practically, the major setup of the power-managing module 16 is to distribute the power. In a charge mode, the first power source P1 from the external power source 3 via the USB port 14 is transmitted to the battery 15. In an operation mode, if the external power source 3 is electrically coupled with the USB port 14, then both the first power source P1 and the secondary power source 151 are simultaneously distributed to the indicator 111, the power element 121 and the plurality of light-emitting elements 122. If the external power source 3 is not electrically coupled with the USB port 14, then only the secondary power source 151 is transmitted to the indicator 111, the power element 121 and the plurality of light-emitting elements 122.

Refer now to FIG. 1 through FIG. 5, where FIG. 5 demonstrates a finger to be cooperated with the massage device of FIG. 1. As shown, when a user manipulates the first embodiment of the massage device 1 in accordance with the present invention, the fastening belt 2 is firstly applied to fix the device body 11 onto the finger 100, by having the transparent protrusive massage bodies 123, 123a and 123b to contact at least the acupuncture point 1001, 1002 and 1003 of the finger 100, respectively. Also, the transparent protrusive massage bodies 123c and 123d and the other two non-labeled transparent protrusive massage bodies are adjusted to contact the corresponding acupuncture points. Then, the trigger element 13 is triggered to have the power-managing module 16 to transmit at least one of the first power source P1 and the secondary power source 151 to the indicator 111, the power element 121 and the plurality of light-emitting elements 122, so that the indicator 111 can be light on, the power element 121 can be energized to move the device body 11, and the light-emitting elements 122 can emit light beams LB to travel along the corresponding light paths L.

In the following description, the transparent protrusive massage bodies 123, 123a and 123b would be elucidated. For the transparent protrusive massage bodies 123c and 123d and the other two non-labeled transparent protrusive massage bodies are resembled to the transparent protrusive massage bodies 123, 123a and 123b, in both structuring and operation, thus details thereabout would be omitted herein. When the device body 11 moves, the transparent protrusive massage bodies 123, 123a and 123b exert mechanical massage onto at least the acupuncture points 1001, 1002 and 1003. Namely, through the vibrations or movement of the device body 11, the transparent protrusive massage bodies 123, 123a and 123b can massage mechanically at the acupuncture point 1001, 1002 and 1003, respectively. On the other hand, the light beams LB passing through the corresponding transparent protrusive massage bodies would also hit on the acupuncture points 1001, 1002 and 1003, respectively, so as to generate the corresponding optical massages.

Practically, since each of the transparent protrusive massage bodies 123, 123a and 123b can be a lens structure, thus light beams LB travelling along the corresponding light paths L can penetrate the corresponding transparent protrusive massage bodies 123, 123a and 123b, and then focus and radiate at the corresponding acupuncture points 1001, 1002 and 1003.

It shall be noted that a focus means upon the light beams LB is applied in this embodiment to strengthen the optical massage performance at the acupuncture points 1001, 1002 and 1003. However, in some other embodiments not shown herein, if the energy of the light beam LB is strong enough, then a scattering means upon the light beam LB can be applied to distribute the energy of the light beam to more acupuncture points. Hence, practically, the determination of the lens structure, for focusing or scattering, is mainly determined by the energy provided by the light-emitting element 122 and the body portions to be massaged.

The aforesaid acupuncture points 1001, 1002 and 1003 are proved to the acupuncture points that can improve the blood circulation around the eyes, so as to alleviate the problem of eye fatigue. By providing the mechanical massage and the optical massage simultaneously in the present invention, the massage performance can be further enhanced.

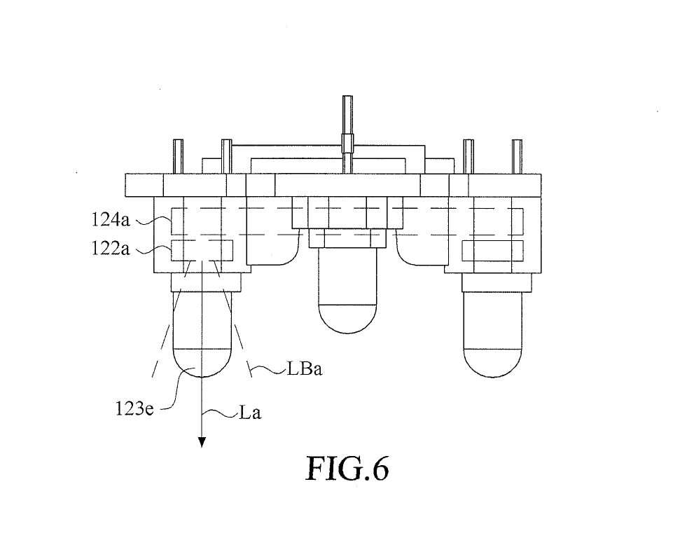

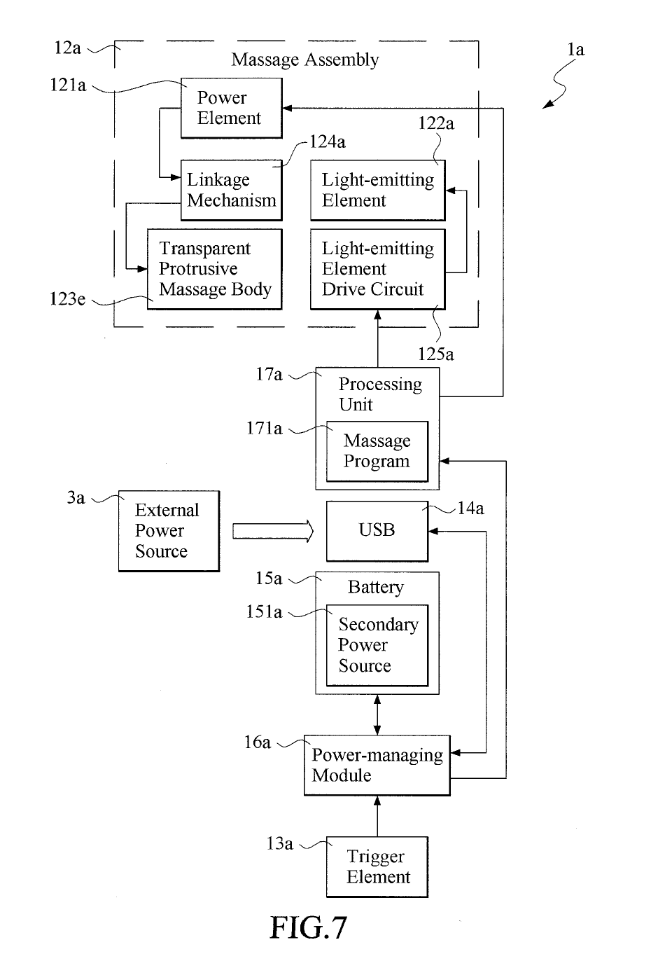

Refer now to FIG. 6 and FIG. 7; where FIG. 6 is a schematic side view of the transparent protrusive massage bodies and the light-emitting elements for a second embodiment of the massage device in accordance with the present invention, and FIG. 7 is a schematic block view of the FIG. 6.

As shown, a major difference between this second embodiment and the aforesaid first embodiment is that, in this second embodiment, the massage assembly 12a includes a power element 121a, at least one light-emitting element 122a, a plurality of transparent protrusive massage bodies 123e, a linkage mechanism 124a and a light-emitting element drive circuit 125a. The linkage mechanism 124a is to connect the power element 121a and the plurality of transparent protrusive massage bodies 123e. The light-emitting element drive circuit 125a is electrically coupled with the plurality of light-emitting elements 122a so as to drive the light-emitting elements 122a to emit light beams LB to travel along the corresponding light paths L.

In addition, the massage device 1a further includes a processing unit 17a located inside the device body (not shown in the figure) and electrically coupled with the power element 121a, the light-emitting element drive circuit 125a, the indicators (not shown in the figure) and the power-managing module 16a. The processing unit 17a further includes a massage program 171a. In the present invention, the processing unit 17a can be a micro controller unit (MCU). In other embodiments, the processing unit can be a processor with relevant management ability. The massage program 171a can be an APP (Application) and, particularly, can include a preset or configurable vibration parameter (such as a vibration frequency, a vibration mode shape or the like) and an illumination parameter (such as a light frequency, a light pattern or the like).

In the present invention, while the second embodiment of the massage device 1a is applied, after the trigger element 13a embodied as a button is triggered, the power-managing module 16a would transmit at least one of the first power source P1 (provided by the external power source 3a) and the secondary power source 151 to the indicator and the processing unit 17a, so as to light up the indicator and to have the processing unit 17a to base on the massage program 171a to activate the light-emitting element drive circuit 125a to further energize the light-emitting elements 122a to emit corresponding light beams LBa to travel along corresponding light paths La. Simultaneously, the power element 121a is also activated to move the device body. In particular, the processing unit 17a can forward at least one of the first power source P1 and the secondary power source 151 to the light-emitting element drive circuit 125a and the power element 121a.

While the power element 121a provides the power, the linkage mechanism 124a moves the transparent protrusive massage bodies 123e synchronously, such that the mechanical massage upon the acupuncture points (not shown in the figure) can be performed. Namely, the transparent protrusive massage body 123e is energized by the power element 121a to exert mechanical massages upon the corresponding acupuncture points. On the other hand, the light beams LBa penetrating the corresponding transparent protrusive massage bodies 123e would exert the optical massages at the corresponding acupuncture points.

Namely, the second embodiment is superior to the first embodiment in its programmability. The second embodiment introduces the processing unit 17a to apply the massage program 171a to control the power element 121a and the light-emitting element drive circuit 125a, such that the power element 121a can have the linkage mechanism 124a to move synchronously the transparent protrusive massage bodies 123e to undergo individual motions. That is to say that each of the transparent protrusive massage bodies 123e can have its own motion pattern to individually exert corresponding mechanical massages upon the respective acupuncture points. Except for the aforesaid details, all other structuring and operations are the same for both the first and the second embodiments.

In summary, by providing the aforesaid massage devices in accordance with the present invention, since the conventional massage blocks have been replaced by the transparent protrusive massage bodies and the light-emitting elements are included to emit individual light beams to travel along the corresponding light paths, so a user of the massage device can apply the transparent protrusive massage bodies to mechanically massage the acupuncture points, and also can optically massage these same acupuncture points via the transparent protrusive massage bodies and the corresponding light-emitting elements. Thereupon, the overall massage effort can be significantly increased.

While the present invention has been particularly shown and described with reference to a preferred embodiment, it will be understood by those skilled in the art that various changes in form and detail may be without departing from the spirit and scope of the present invention.

What is claimed is:

1. A massage device, comprising:
a device body for sleeving one of a finger and a toe; and
a massage assembly, located at the device body, further including:
a power element, located inside the device body to provide a power for moving the device body;
a plurality of light-emitting elements, each of the light-emitting elements being to emit a light beam to travel along a light path; and
a plurality of transparent protrusive massage bodies, each of the transparent protrusive massage bodies being located on the corresponding light path of the light beam emitted by the corresponding light-emitting element;
wherein, when the device body sleeves one of the finger and the toe, the plurality of transparent protrusive massage bodies contact the one of the finger and the toe at one acupuncture point at least;
wherein, when the device body is moved, the plurality of transparent protrusive massage bodies massage mechanically the at least one acupuncture point, and the light beam penetrates the corresponding transparent protrusive massage body to massage optically the at least one acupuncture point.

2. The massage device of claim 1, wherein the transparent protrusive massage body includes a lens structure.

3. The massage device of claim 1, wherein the light-emitting element is an LED (light-emitting diode).

4. The massage device of claim 1, wherein the massage assembly further includes a light-emitting element drive circuit electrically coupled with the light-emitting elements to have the light-emitting elements to emit the corresponding light beams.

5. The massage device of claim 4, further including a processing unit located inside the device body and electrically coupled with the power element and the light-emitting element drive circuit, the processing unit further including a massage program, the processing unit basing on the massage program to trigger the light-emitting element drive circuit to have the light-emitting elements to emit the light beams and to have the power element to provide a power for moving the device body.

6. The massage device of claim 5, wherein the processing unit is a micro controller unit (MCU).

7. The massage device of claim 5, further including:
a USB (Universal serial bus) port, located at the device body, electrically coupled with an external power source for providing a first power source;
a battery, located inside the device body, storing a secondary power source; and
a power-managing module, located inside the device body, electrically coupled with the USB port, the battery and the processing unit, being to transmit at least one of the first power source and the secondary power source to the processing unit, being further to transmit the first power source to the battery to be stored as the secondary power source.

8. The massage device of claim 7, further including a trigger element located at the device body and electrically coupled with the power-managing module; wherein, when the trigger element is triggered, the power-managing module transmits at least one of the first power source and the secondary power source to the processing unit, so that the processing unit triggers the light-emitting element drive circuit to have the light-emitting elements to emit the light beams and to have the power element to move the device body.

9. The massage device of claim 8, wherein the trigger element is a button.

10. The massage device of claim 5, wherein the device body includes a plurality of indicators electrically coupled with the processing unit, the processing unit basing on the massage program to control the indicators.

11. The massage device of claim 1, wherein the device body has two through holes located individually to two opposing sides of the device body, the through hole allowing a fastening belt to pass therethrough so as thereby to fix the device body onto one of the finger and the toe, such that the transparent protrusive massage bodies are able to contact the at least one acupuncture point of the one of the finger and the toe.

12. The massage device of claim 1, wherein the power element is a motor.

13. The massage device of claim 1, further including:
a USB (Universal serial bus) port, located at the device body, electrically coupled with an external power source for providing a first power source;
a battery, located inside the device body, storing a secondary power source; and
a power-managing module, located inside the device body, electrically coupled with the USB port, the battery and the processing unit, being to transmit at least one of the first power source and the secondary power source to the processing unit, being further to transmit the first power source to the battery to be stored as the secondary power source.

14. The massage device of claim 13, further including a trigger element located at the device body and electrically coupled with the power-managing module; wherein, when the trigger element is triggered, the power-managing module transmits at least one of the first power source and the secondary power source to the power element and the light-emitting elements.

15. The massage device of claim 14, wherein the trigger element is a button.

16. The massage device of claim 14, wherein the device body further includes a plurality of indicators electrically coupled with the power-managing module; wherein, when the trigger element is triggered, the power-managing module further transmits at least one of the first power source and the secondary power source to the indicators so as to light up the indicators.

17. The massage device of claim 1, wherein the massage assembly further includes a linkage mechanism connecting the power element and the transparent protrusive massage bodies; wherein, when the power element provides the power, the linkage mechanism moves the transparent protrusive massage bodies synchronously.
